(19) United States
(12) Patent Application Publication
(10) Pub. No.: US 2026/0354358 A1
(43) Pub. Date: Nov. 12, 2026

(54) Methods and Materials for the Conditional Hepatic Expression of Transgenes in Mammalian Bioincubators

(71) Applicant: Cytotheryx, Inc., Rochester, MN (US)

(72) Inventors: John Swart, Orange City, IA (US); Ryan Schulze, Rochester, MN (US); Brian Dacken, Sheldon, IA (US); Brent Pepin, Rochester, MN (US); Kari Allen, Rochester, MN (US)

(21) Appl. No.: 19/118,390

(22) PCT Filed: Oct. 6, 2023
(86) PCT No.: PCT/US2023/076266

§ 371 Date: Apr. 4, 2025

Related U.S. Application Data
(60) Provisional application No. 63/414,246, filed on Oct. 7, 2022.

(51) Int. Cl. A01K 67/0275; C12N 9/14

(52) U.S. Cl. CPC A01K 67/0275; C12N 9/14; C12Y 307/01002; A01K 2217/203; A01K 2217/206; A01K 2227/105; A01K 2227/108

(57) ABSTRACT

Transgenic bioincubators are provided having an FAH transgene or endogenous FAH modified with an inducible promoter. The inducible promoter allows for control of the expression of FAH in the presence or absence of an inducer, for purposes of generating conditional inducibility of the FAH transgene or endogenous FAH. This allows for the controlled expression of FAH only when desired for purposes of improving the health of the bioincubator during the process of primary human hepatocyte expansion.

### CROSS REFERENCE TO RELATED APPLICATION(S)

This application claims priority to International PCT Application No. PCT/US23/76266, filed on Oct. 6, 2023, which claims priority to and the benefit under 35 U.S.C. § 119(e) to U.S. Provisional Application No. 63/414,246, filed Oct. 7, 2022, entitled “Methods and Materials for the Conditional Hepatic Expression of Transgenes in Mammalian Bioincubators,” which is hereby incorporated by reference in its entirety for all purposes.

### TECHNICAL FIELD

The disclosure relates to bioincubators with tight and titratable expression of an fumarylacetoacetate hydrolase (FAH) transgene and uses thereof. This disclosure also relates to methods of expanding hepatocytes from other species in animal bioincubators, including pigs, rats, and mice.

### BACKGROUND

An impediment to the utilization of primary human hepatocytes (HHCs) for the treatment of liver disease is the poor availability of a consistent source and large supply of clinically safe cells. As HHCs are unable to be propagated ex vivo, specialized “bioincubators” have been engineered to harness the regenerative environment of the mammalian liver for purposes of HHC expansion.

One such bioincubation system comprises immunodeficient animals (Rag2/IL2r-Gamma knockouts) harboring a disruption in the gene encoding fumarylacetoacetate hydrolase (FAH knockout), an enzyme involved in the catabolismof the amino acid tyrosine. Such ‘FRG’ bioincubators are susceptible to progressive acute liver failure due to a buildup of hepatotoxic tyrosine degradation intermediates, including fumarylacetoacetate and succinylacetone. This damage can be effectively circumvented by administration of nitisinone/NTBC, which blocks tyrosine degradation upstream of FAH and therefore precludes fumarylacetoacetate and succinylacetone formation. Xenotransplant of healthy HHCs into the FRG bioincubator is therefore possible by virtue of: i) the immunodeficient phenotype of the FRG bioincubator and ii) the selective growth advantage afforded by the HHC-provided copy of FAH. By cycling an FRG animal on/off of NTBC, HHCs can replace damaged endogenous hepatocytes and, importantly, expand significantly within the bioincubator. At high levels of repopulation, NTBC is no longer required and the expanded HHCs effectively render the FRG animal ‘cured’ of its FAH deficiency.

However, this model is associated with drawbacks. The model necessitates the use of expensive nitisinone/NTBC therapy throughout gestation and at early stages of engraftment to ensure animal viability. In large animal (e.g., pig) FRG bioincubators, such as those disclosed in U.S. Pat. No. 9,485,971, this may require months of NTBC support during gestation and an additional 3 months or more of NTBC cycling during the early stages of HHC engraftment and expansion within FRG animals. Furthermore, although NTBC therapy can prevent the outward phenotype of FAH deficiency, there is evidence that extended NTBC usage can result in developmental and cognitive impairment in humans (see e.g., Garcia, M. I., et al. (2017). “Long-term cognitive functioning in individuals with tyrosinemia type 1 treated with nitisinone and protein-restricted diet.” Mol Genet Metab Rep 11:12-16). By blocking 4-hydroxyphenylpyruvate dioxygenase (HPD), NTBC therapy results in the accumulation of tyrosine in treated animals, effectively resulting in a phenotype equivalent to a rare metabolic disorder, hereditary tyrosinemia type 3 (HT3). Importantly, human patients with HT3 have demonstrated neurological symptoms that include intellectual disability, seizures, and ataxia (Cerone, R., et al. (1997). “Tyrosinemia type III: diagnosis and ten-year follow-up.” Acta Paediatr 86(9): 1013-1015). Animals treated for extended periods of time with NTBC, especially during gestation, may therefore be susceptible to similar neurological manifestations of the HT3 phenotype.

There is a need in the art for improved specialized bioincubators.

### SUMMARY

In Example 1, a transgenic bioincubator has a genome comprising an FAH transgene or endogenous FAH modified with an inducible promoter, wherein the inducible promoter allows for control of the expression of FAH in the presence or absence of an inducer.

Example 2 relates to the transgenic bioincubator of Example 1, wherein the transgenic bioincubator is a pig, mouse, or rat.

Example 3 relates to the transgenic bioincubator of Examples 1 or 2, wherein the inducible promoter is a hepatocyte-specific promoter.

Example 4 relates to the transgenic bioincubator of any one of Examples 1 to 3, wherein the inducible promoter comprises porcine albumin promoter (P_{alb}), thyroxine-binding globulin promoter (P_{TBG}), minimal transthyretin promoter (P_{TTR}), cytomegalovirus promoter (CMV), or CMV early enhancer/chicken β-actin promoter (CAG).

Example 5 relates to the transgenic bioincubator of any one of Examples 1 to 4, wherein the endogenous FAH is modified by incorporation of the inducible promoter at an endogenous locus.

Example 6 relates to the transgenic bioincubator of any one of Examples 1 to 5, wherein the inducer comprises tetracycline, doxycycline, cumate, veledimex, FKCsA, abscisic acid (ABA), or 4-hydroxytamoxifen (4-OHT).

Example 7 relates to the transgenic bioincubator of any one of Examples 1 to 6, wherein the bioincubator is an immunodeficient FRG animal or immunodeficient RG animal.

In Example 8, a method of regulating and/or controlling the expression of FAH in a transgenic bioincubator comprises providing an immunocompromised transgenic bioincubator comprising an FAH transgene or endogenous FAH modified with an inducible promoter, and administering to the transgenic bioincubator an inducer for regulating and/or controlling the expression of FAH in the presence or absence of the inducer.

Example 9 relates to the method of Example 8, wherein the expression of FAH is inducible in hepatocytes.

Example 10 relates to the method according to Example 8 or 9, wherein the transgenic bioincubator is a pig, mouse, or rat.

Example 11 relates to the method according to any one of Examples 8 to 10, wherein the inducible promoter is a hepatocyte-specific promoter.

Example 12 relates to the method according to any one of Examples 8 to 11, wherein the inducible promoter comprises porcine albumin promoter (P_{alb}), thyroxine-binding globulin promoter (P_{TBG}), minimal transthyretin promoter (P_{TTR}), cytomegalovirus promoter (CMV), or CMV early enhancer/chicken β-actin promoter (CAG).

Example 13 relates to the method according to any one of Examples 8 to 12, wherein the endogenous FAH is modified by incorporation of the inducible promoter at an endogenous locus.

Example 14 relates to the method according to any one of Examples 8 to 13, wherein the inducer comprises tetracycline, doxycycline, cumate, veledimex, FKCsA, abscisic acid (ABA), or 4-hydroxytamoxifen (4-OHT).

Example 15 relates to the method according to any one of Examples 8 to 14, wherein the bioincubator is an immunodeficient FRG animal or immunodeficient RG animal.

### BRIEF DESCRIPTION OF THE FIGURES

FIG. 1 shows a diagram of a bidirectional Tet-On system for control of FAH expression in pigs.

FIG. 2 shows a diagram of inducible FAH expression in hepatocytes using a cumate switch system.

FIG. 3 shows a diagram of inducible FAH expression in hepatocytes using a RheoSwitch system.

FIG. 4 shows a diagram of Cre-recombinase-mediated excision of FAH in hepatocytes.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

### Terms and Definitions

The embodiments of this disclosure are not limited to particular bioincubators with expression of an FAH transgene, which can vary and are understood by skilled artisans. It is further to be understood that all terminology used herein is for the purpose of describing particular embodiments only, and is not intended to be limiting in any manner or scope. So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below. All units, prefixes, and symbols may be denoted in its SI accepted form.

The term “about,” as used herein, refers to variation in the numerical quantity that can occur, for example, through typical measuring techniques and equipment, with respect to any quantifiable variable, including, but not limited to, mass, volume, time, distance, wavelength, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “administer” or “administration,” as used herein, refers to providing or giving a subject an agent, such as a therapeutic agent, by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

As used herein, the term “disruption” in a gene refers to any insertion, deletion or point mutation, or any combination thereof. In some embodiments, the disruption leads to a partial or complete loss of expression of mRNA and/or functional protein. In other embodiments, the disruption may occur at a location outside of a given gene, to prevent expression of said given gene.

The term “engraft” or “engraftment,” as used herein, refers to implanting cells or tissues in an animal. As used herein, engraftment of human hepatocytes in a recipient animal refers to the process of human hepatocytes becoming implanted in the recipient animal following injection. Engrafted human hepatocytes are capable of expansion in the recipient animal.

The term “expand” or “expansion,” as used herein, refers to an increase in quantity. As used herein, “expanding” human hepatocytes refers to the process of allowing cell division to occur such that the number of human hepatocytes increases.

The term “fumarylacetoacetate hydrolase (FAH),” as used herein, refers to a metabolic enzyme that catalyzes the last step of tyrosine catabolismaH as used herein refers to all forms of FAH in mammals. Mammals having a homozygous deletion of the FAH gene exhibit altered liver mRNA expression and severe liver dysfunction. Point mutations in the FAH gene have also been shown to cause hepatic failure and postnatal lethality (Aponte et al. Proc. Natl. Acad. Sci. U.S.A. 98(2): 641-645, 2001). Humans deficient for FAH develop the liver disease hereditary tyrosinemia type 1 (HT1) and, without intervention, develop liver failure. FAH deficiency leads to accumulation of fumarylacetoacetate, a potent oxidizing agent, ultimately leading to cell death of hepatocytes deficient for FAH. Thus, FAH-deficient animals can be repopulated with hepatocytes from other species, including humans.

The term “gestation,” as used herein, refers to the period of development from conception (fertilization of an oocyte) to birth. The gestation period for a pig is (on average) 112-115 days.

The term “hepatocyte,” as used herein, refers to a type of cell that makes up 70-80% of the cytoplasmic mass of the liver. Hepatocytes are involved in protein synthesis, protein storage and transformation of carbohydrates, synthesis of cholesterol, bile salts and phospholipids, and detoxification, modification, and excretion of exogenous and endogenous substances. The hepatocyte also initiates the formation and secretion of bile. Hepatocytes manufacture serum albumin, fibrinogen and the prothrombin group of clotting factors and are the main site for the synthesis of lipoproteins, ceruloplasmin, transferrin, complement and glycoproteins. In addition, hepatocytes have the ability to metabolize, detoxify, and inactivate exogenous compounds such as drugs and insecticides, and endogenous compounds such as steroids.

The term “IL2r-Gamma,” as used herein, refers to the common-y chain of the interleukin receptor (I12rg), a gene encoding the common gamma chain of interleukin receptors. IL2rg is a component of the receptors for a number of interleukins, including IL-2, IL-4, IL-7 and IL-15. Animals deficient in IL2rg exhibit a reduction in B cells and T cells and lack natural killer cells. The term is also known as interleukin-2 receptor gamma chain.

The term “immunodeficient,” as used herein, refers to lacking in at least one essential function of the immune system. As used herein, an “immunodeficient” animal is one lacking in specific components of the immune system or lacking function of specific components of the immune system (such as, for example, B cells, T cells or NK cells). In some cases, an immunodeficient animal lacks macrophages. In some embodiments, an immunodeficient animal comprises one or more genetic alterations that prevent or inhibit the development of functional immune cells (such as B cells, T cells or NK cells). In some examples, the genetic alteration is in the Rag1, Rag2 or IL2rg gene. Thus, in some cases, the immunodeficient animal is Rag1^{-/-}, Rag2^{-/-} or IL2^{-/-}.

The term “NTBC” or (2-nitro-4-trifluoro-methyl-benzoyl)-1,3 cyclohexanedione), as used herein, refers to an inhibitor of 4-hydroxy-phenylpyruvate dioxygenase (HPPD). HPPD catalyzes the conversion of 4-hydroxyphenylpyruvate to homogentisate, the second step in tyrosine catabolismoreatment with NTBC blocks the tyrosine catabolismpathway at this step and prevents the accumulation of fumarylacetoacetate and succinylacetone, a pathognomonic metabolite that accumulates in FAH-deficient humans and animals.

The term “Rag2” or “recombinase activating gene 2,” as used herein, refers to a gene involved in recombination of immunoglobulin and T cell receptor loci. Animals deficient in the Rag2 gene are unable to undergo V(D)J recombination, resulting in a complete loss of functional T cells and B cells.

The term “transgene,” as used herein, refers to an exogenous nucleic acid sequence introduced into a cell or the genome of an organism.

The term “transgenic animal,” as used herein, refers to a non-human animal, usually a mammal, having a non-endogenous (heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal. A “transgene” is meant to refer to such heterologous nucleic acid, such as, heterologous nucleic acid in the form of an expression construct (such as for the production of a “knock-in” transgenic animal) or a heterologous nucleic acid that upon insertion within or adjacent to a target gene results in a decrease in target gene expression (such as for production of a “knock-out” transgenic animal). A “knock-out” of a gene means an alteration in the sequence of the gene that results in a decrease of function of the target gene, preferably such that target gene expression is undetectable or insignificant. Transgenic knock-out animals can comprise a heterozygous knock-out of a target gene, or a homozygous knock-out of a target gene. “Knock-outs” also include conditional knock-outs, where alteration of the target gene can occur upon, for example, exposure of the animal to a substance that promotes target gene alteration, introduction of an enzyme that promotes recombination at the target gene site (for example, Cre in the Cre-lox system), or other method for directing the target gene alteration postnatally.

The term “transplant” or “transplanting,” as used herein, refers to the process of grafting an organ, tissue, or cells from one subject to another subject, or to another region of the same subject.

The term “safe harbor locus,” as used herein, refers to a genomic site that when a transgene is integrated, there would be no change in cellular behavior except that determined by the new transgene.

There remains a need for specialized bioincubators engineered to harness the regenerative environment of the mammalian liver for purposes of HHC expansion. In further embodiments, there remains a need for the restoration of wild-type FAH functionality within a bioincubator.

To improve overall animal health and to avoid potential complications arising from long-term NTBC usage, including, for example, in newborn FRG piglets, it is desirable in some embodiments to allow for the temporal expression of wild-type FAH during the 120+ days of gestation and restoration of the wild-type FAH at intermittent periods during the HHC cell engraftment period. In embodiments, the present disclosure provides methods and processes for accomplishing this in FRG animals by introduction of a full-length copy of wild-type FAH under the control of an inducible promoter. In some embodiments, fine-tuning may be achieved for spatiotemporal control by including elements restricting FAH expression to hepatocytes, using the albumin (P_{Alb}) or thyroxine-binding globulin (P_{TBG}) promoters, for example, to ensure specificity. Higher levels of FAH expression may be achieved in other embodiments by using the cytomegalovirus (CMV) or CMV immediate-early enhancer/chicken beta-actin (CAG) promoters for inducible expression of the FAH transgene. In other embodiments, immunodeficient ‘RG’ animals may be used as a starting template, whereupon the native FAH promoter may be modified by incorporation of an inducible promoter at its endogenous locus.

In some aspects, disclosed herein is the generation of bioincubators with tight and titratable expression of an FAH transgene. The disclosure further relates to the generation of a stably inserted transgene that accompanies existing FRG (FAH/Rag2/IL2r-Gamma) mutations, in order to provide for the controlled and inducible expression of wild-type FAH. Beneficially, the ability to control and regulate the expression of FAH via an FAH transgene can reduce complications associated with long term administration of NTBC, or a similar compound. In some embodiments, the bioincubators are animal FRG bioincubators. In further embodiments, the bioincubators are immunodeficient (i.e., RG) bioincubators wherein FAH expression is conditionally expressed at its native locus. In some aspects, the bioincubator is a pig, mouse, or rat. In further aspects, the bioincubator is a pig. Provided herein are various methods for regulating and controlling the expression of FAH in an immunodeficient bioincubator.

In embodiments, inducible gene editing systems are useful for temporally-controlled, spatially-controlled, and both temporally-controlled and spatially-controlled modification of genes. Some embodiments utilize a Tetracycline-Inducible System, including the Tet-Off or Tet-On inducible system. They are now commonly referred to as the Tet-Off system (tTA dependent) and the Tet-On system (rtTA dependent). In each system, a recombinant tetracycline-controlled transcription factor, tetracycline-controlled transactivator (tTA) or reverse tetracycline-controlled transactivator (rtTA), interacts with a tTA/rtTA responsive promoter, Ptet, to control expression of the gene of interest. Expression is regulated by the effector substance tetracycline (Tc) or one of its derivatives. In embodiments, Tet-On systems respond to doxycycline (Dox). Tetracyclines act at the level of DNA binding of tTA and rtTA transcription factors. In aspects, rtTA requires a tetracycline ligand for DNA binding and transcription, and by contrast, the interaction between tTA and DNA is prevented by tetracycline. Thus, the two versions of the Tet system respond to tetracyclines differently and may be used in a complementary manner.

tTA is a hybrid transcription factor resulting from the fusion of the prokaryotic Tet repressor, TetR, with a eukaryotic transcriptional transactivation domain (e.g., HSV VP16). The TetR moiety confers sequence specific DNA binding, sensitivity to tetracyclines and dimerization to the tTA fusion protein. Accordingly, the response of both TetR and tTA to tetracyclines is similar: binding of the antibiotic lowers their affinity to their common cognate binding sites, the tet operators. rtTA differs from tTA by a few point mutations within TetR. These, however, result in a complete reversal of tetracycline responsiveness of this transcription factor. rtTA requires tetracyclines for binding to a tetracycline operator sequence (tetO). Specific tetracycline derivatives such as doxycycline (Dox) or anhydrotetracycline (ATc) may be used to exploit the rtTA phenotype.

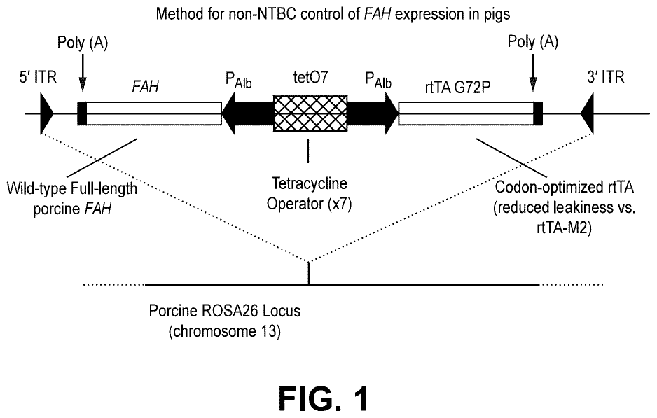

In some embodiments, a bidirectional Tet-On system with both a codon-optimized rtTA and FAH are each under the control of a promoter, including but not limited to, the porcine albumin promoter (P_{Alb}), thyroxine-binding globulin (P_{TBG}) promoter, or a minimal transthyretin promoter (TTR), and downstream of a Tet operator (TetO) containing an array of TetO sequences. An example of a bidirectional Tet-On system is shown in FIG. 1. The use of the various hepatocyte-specific promoters can drive rtTA and FAH expression. In some embodiments, the hepatocyte-specific promoter may only drive rtTA expression. In further embodiments, a stronger promoter, including but not limited to CMV, may be used to drive FAH expression following doxycycline administration.

The system can allow for doxycycline-inducible expression of FAH throughout gestation of an FRG bioincubator. Such a gene cassette can be placed within a safe harbor locus of the genome of the bioincubator to minimize concerns regarding unwanted gene disruption due to insertional mutagenesis. In some embodiments, the genome is the Sus scrofa genome (i.e., ROSA26 on chromosome 13). In further embodiments, the safe harbor locus comprises AAVS1, ROSA26, COL1A1, H11, ACTB, GAPDH, or Pifs501. Furthermore, the rtTA used may take advantage of single-amino acid substitutions (e.g., Gly72Pro) to reduce leakiness of FAH expression to near-undetectable levels.

In other embodiments, the inducible expression of FAH can be regulated by a cumate switch system, in which CymR repressor, a key regulator of cysteine metabolism, is placed under the control of a hepatocyte-specific promoter. Beneficially, cumate is an inexpensive and non-toxic small-molecule inducer. In aspects, the hepatocyte-expressed CymR repressor binds tightly to cumate operator (CymO) sequences upstream of FAH in the absence of cumate. Upon administration of cumate to the animal, the high affinity of this inducer for CymR results in the release of repression at these sites, permitting FAH expression.

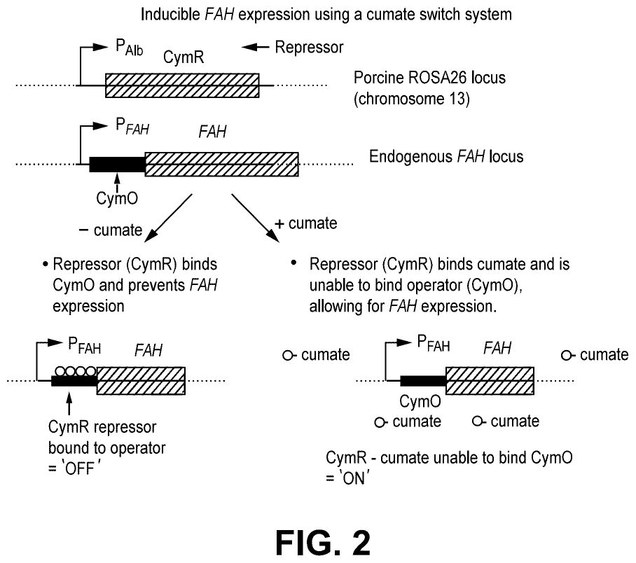

An example of the cumate switch system is shown in FIG. 2. Within FIG. 2, the gene encoding the CymR repressor from Bacillus subtilis is placed under the control of the albumin promoter at a safe harbor site in the Sus scrofa genome, to provide hepatocyte-specific expression. Under basal conditions, CymR is free to bind to the CymO operator sequence that has been engineered upstream of the endogenous FAH locus. This tight binding prevents FAH expression under normal conditions. When cumate is administered to the animal, it binds to and causes a conformational change in CymR that prevents its ability to bind DNA, thereby relieving repression of FAH. This system can be modified to instead take advantage of alternative hepatocyte-specific promoters, including but not limited to PTBG, or to engineer an inducible FAH transgene at a non-endogenous locus.

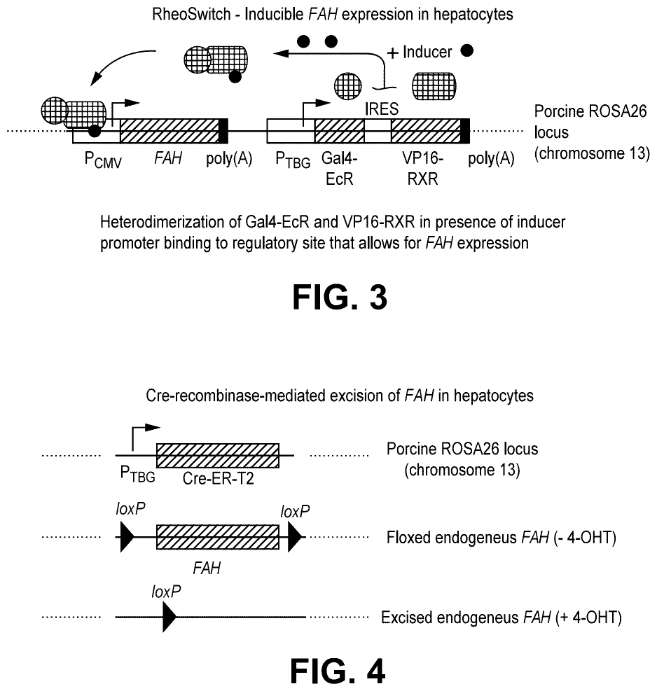

In further embodiments, it can be advantageous to use a protein-protein interaction-based chimeric system, where the FAH transgene can be placed under the inducible control of a RheoSwitch, which allows for extremely tight regulation of gene expression. In embodiments, the constitutive expression of Gal4-EcR and VP16-RXR fusion proteins is driven by a hepatocyte-specific promoter. Heterodimerization of these fusion proteins in the presence of a small molecule diacylhydrazine ligand (veledimex) activates FAH expression from a responsive promoter. An example of the RheoSwitch system is shown in FIG. 3. In FIG. 3, two genes, VP16-RXR (VP16 transcription activation domain fused to the EF domain of a chimeric RXR) and Gal4-EcR (Gal4 DNA-binding domain fused to the ecdysone receptor) are placed under the control of a hepatocyte-specific promoter, PTBG. Administration of an inducer (veledimex) promotes heterodimerization of the gene products and allows for their stable association upstream of an FAH transgene engineered into the safe harbor locus on chromosome 13 of the porcine genome. Upon binding, this complex induces strong expression of FAH. Removal of the inducer results in rapid dissociation of the complex and a return to the basal state of expression. This strategy can be modified in a number of ways; for instance, through the use of i) FKBP12-Gal4DBD/Cyclophilin-VP16; or ii) ABI1-Gal4DBD/PYL1-VP16 partnerships, which would promote the subsequent expression of FAH only in the presence of FKCsA or abscissic acid (ABA), respectively.

In still further embodiments, FAH can be placed downstream of an upstream activation sequence, the Gal4DBD binding site. In aspects, the hepatocyte-specific expression of FKBP12-Gal4DBD and Cyclophilin-VP16 fusion peptides restrict binding to Gal4DBD and promote the subsequent expression of FAH only in the presence of the non-toxic and non-immunosuppressive synthetic compound FKCsA. A modification of this protein-protein interaction based system might instead use ABI1-Gal4DBD and PYL1-VP16 as the fusion peptides, in which case the non-toxic abscissic acid (ABA) can be administered to facilitate heterodimerization and downstream FAH expression.

In yet further embodiments, FAH might be excised using an inducible Cre/loxP system. In some aspects, it can be advantageous to promote the knockout of FAH when its expression is no longer required for the purposes of animal health or HHC expansion. In some embodiments, the expression of CreER-T2 driven by a hepatocyte-specific promoter can be activated upon exogenous administration of 4-hydroxytamoxifen (4-OHT) to the animal. Binding of 4-OHT to CreER-T2 can result in its translocation into the nucleus of hepatocytes, whereupon it may act either on floxed copies of an FAH transgene present in an FRG bioincubator, or on floxed copies of the endogenous FAH gene in an RG bioincubator. In both cases, the recombinase activity results in the excision of the loxP-flanked sequences, leading to permanent and unrecoverable inactivation of FAH.

An example of excising FAH is shown in FIG. 4. As shown in FIG. 4, tamoxifen-inducible Cre can be fused to a modified estrogen receptor (ER-T2) and placed under the control of a hepatocyte-specific promoter (e.g., PTBG). Using CRISPR/Cas9 technology, for example, it can be feasible to engineer loxP sites on either end of the endogenous FAH locus, such that when FAH expression is no longer desired, the animal is treated with 4-hydroxytamoxifen (4-OHT) by intraperitoneal injection. This results in translocation of Cre-ER-T2 to the nucleus of the hepatocytes, where recombination and excision of native FAH occurs.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

The disclosures being thus described, it will be obvious that the same may be varied in many ways. Such variations are not to be regarded as a departure from the spirit and scope of the disclosures and all such modifications are intended to be included within the scope of the following claims.

What is claimed is:

1. A transgenic bioincubator having a genome comprising:
an FAH transgene or endogenous FAH modified with an inducible promoter,
wherein the inducible promoter allows for control of the expression of FAH in the presence or absence of an inducer.

2. The transgenic bioincubator of claim 1, wherein the transgenic bioincubator is a pig, mouse, or rat.

3. The transgenic bioincubator of claim 1 or 2, wherein the inducible promoter is a hepatocyte-specific promoter.

4. The transgenic bioincubator of any one of claims 1 to 3, wherein the inducible promoter comprises porcine albumin promoter (P_{alb}), thyroxine-binding globulin promoter (P_{TBG}), minimal transthyretin promoter (P_{TTR}), cytomegalovirus promoter (CMV), or CMV early enhancer/chicken β-actin promoter (CAG).

5. The transgenic bioincubator of any one of claims 1 to 4, wherein the endogenous FAH is modified by incorporation of the inducible promoter at an endogenous locus.

6. The transgenic bioincubator of any one of claims 1 to 5, wherein the inducer comprises tetracycline, doxycycline, cumate, veledimex, FKCsA, abscisic acid (ABA), or 4-hydroxytamoxifen (4-OHT).

7. The transgenic bioincubator of any one of claims 1 to 6, wherein the bioincubator is an immunodeficient FRG animal or immunodeficient RG animal.

8. A method of regulating and/or controlling the expression of FAH in a transgenic bioincubator comprising:
providing an immunocompromised transgenic bioincubator comprising an FAH transgene or endogenous FAH modified with an inducible promoter; and
administering to the transgenic bioincubator an inducer for regulating and/or controlling the expression of FAH in the presence or absence of the inducer.

9. The method of claim 8, wherein the expression of FAH is inducible in hepatocytes.

10. The method of claim 8 or 9, wherein the transgenic bioincubator is a pig, mouse, or rat.

11. The method of any one of claims 8 to 10, wherein the inducible promoter is a hepatocyte-specific promoter.

12. The method of any one of claims 8 to 11, wherein the inducible promoter comprises porcine albumin promoter (P_{alb}), thyroxine-binding globulin promoter (P_{TBG}), minimal transthyretin promoter (P_{TTR}), cytomegalovirus promoter (CMV), or CMV early enhancer/chicken β-actin promoter (CAG).

13. The method of any one of claims 8 to 12, wherein the endogenous FAH is modified by incorporation of the inducible promoter at an endogenous locus.

14. The method of any one of claims 8 to 13, wherein the inducer comprises tetracycline, doxycycline, cumate, veledimex, FKCsA, abscisic acid (ABA), or 4-hydroxytamoxifen (4-OHT).

15. The method of any one of claims 8 to 14, wherein the bioincubator is an immunodeficient FRG animal or immunodeficient RG animal.
